(19) United States
(12) Patent Application Publication
(10) Pub. No.: US 2013/0084312 A1
(43) Pub. Date: Apr. 4, 2013

(54) NANOPARTICLES FOR DELIVERY OF BIOACTIVE AGENTS

(71) Applicants: Gregory A. CAPUTO, Glassboro, NJ (US); Yaquan SUN, Menands, NY (US); Lei YU, Cherry Hill, NJ (US); Sireesh APPAJOSYULA, Union, NJ (US)

(72) Inventors: Gregory A. CAPUTO, Glassboro, NJ (US); Yaquan SUN, Menands, NY (US); Lei YU, Cherry Hill, NJ (US); Sireesh APPAJOSYULA, Union, NJ (US)

(73) Assignee: ROSE PHARMACEUTICALS, LLC, Boca Raton, FL (US)

(21) Appl. No.: 13/494,953

(22) Filed: Jun. 12, 2012

Related U.S. Application Data
(60) Provisional application No. 61/496,491, filed on Jun. 13, 2011.
(60) Provisional application No. 61/501,673, filed on Jun. 27, 2011.

(51) Int. Cl. A61K 47/34; C08G 63/91; C08G 63/40

(52) U.S. Cl. CPC A61K 47/34; C08G 63/40; C08G 63/916; Y10S 977/773
USPC 424/400; 514/363; 514/21.7; 528/307; 514/772.3; 525/437; 977/773

(57) ABSTRACT

Biocompatible polymeric nanoparticles for delivery of bioactive agents, and methods for preparing the particles, are described. Polyoxalate nanoparticles of the subject technology show desired particle sizes suitable for use in drug delivery and a substantially uniform or narrow particle size distribution. The polyoxalate nanoparticles can contain water-soluble, poorly water-soluble, or water-insoluble drugs. The nanoparticles are nontoxic and are generally safe for use in humans. After being administered into the body, the nanoparticles with a high content of a bioactive agent entrapped therein can safely deliver the agent to target sites and stably release the drug at a controlled rate.

### RELATED APPLICATIONS

This application claims the priority benefit of U.S. Provisional Application No. 61/496,491, filed Jun. 13, 2011, and U.S. Provisional Application No. 61/501,673, filed Jun. 27, 2011. The contents of each of the foregoing applications are hereby incorporated by reference herein in their entireties.

### FIELD

The subject technology relates to a method for preparing biocompatible polymeric nanoparticles for use in drug delivery. More specifically, the subject technology relates to biocompatible polymeric nanoparticles made up of polyoxalate polymers, which are nontoxic and safe to the body, and which can be used for drug delivery.

### BACKGROUND

Currently, there is enormous interest in using nanotechnology for a variety of applications, including biomedical ones. Nanoparticles offer many advantages when used for applications such as the delivery of bioactive agents (e.g., DNA, AIDS drugs, immunosuppressants, chemotherapeutics), and drug uptake and degradation (e.g., enzyme encapsulation). In addition, nanotechnology offers considerable potential when applied in other areas, such as agricultural processes (e.g., plant genetics; or controlled fertilizer and pesticide release using sensor technology linked to the payload release mechanism), industrial synthetic processes, and environmental applications (e.g. ultra-effective decontamination and disinfection of areas contaminated with toxic chemicals and/or biotoxins such as bacteria or viruses).

In the biomedical area, there are currently a number of nanoparticle systems that have been developed for drug delivery such as the polymeric micelles which are formed from block copolymers consisting of hydrophilic segments and hydrophobic segments. Most of the previously documented block copolymers, however, have not been deemed safe for use in the body, and entail many problems upon clinical application.

Other nanoparticle-based drug delivery systems that have been widely investigated are those involving of synthetic biodegradable polymers such as poly-c-caprolactone and polylactide due to their good biocompatibility. However, these nanoparticles are not ideal carriers for hydrophilic drugs because of their hydrophobic property.

Generally, factors that arc related to the use of particle-based delivery systems in the biomedical area include the particle structure and size; surface properties; biocompatibility; toxicity; biodegradability; and the mechanism and timing of release of the payload from the particle. Other factors, such as the cost of particle formulation and payload loading, the ease of manufacture, and the ability to recover unloaded payload are also important.

Hence, despite the availability of a number of nanoparticle drug delivery systems, there is a continuing need for a new system for the delivery of drugs and other payloads.

### SUMMARY

The subject technology is illustrated, for example, according to various aspects described below. Various examples of aspects of the subject technology are described as numbered clauses (1, 2, 3, etc.) for convenience. These are provided as examples and do not limit the subject technology. It is noted that any of the dependent clauses may be combined in any combination, and placed into a respective independent clause, e.g., clause 1 or clause 2. The other clauses can be presented in a similar manner.

1. Polyoxalate nanoparticles having diameters ranging from about 50 to about 300 nm.

2. A method for preparing biocompatible nanoparticles of polyoxalate polymers, comprising:
(a) mixing 1,4-cyclohexanedimethanol (CDE) with oxalyl chloride (OC) in the presence of dichloromethane (DCM), and triethylamine to produce polyoxalate polymer; and
(b) adding the polyoxalate polymer to a solution of polyvinylalcohol (PVA) in the presence of a suitable solvent to produce a substantially homogenous emulsion of polyoxalate nanoparticles.

3. The method of clause 2, wherein the produced polyoxalate nanoparticles have diameters ranging from about 50 to about 300 nm.

4. The method of clause 2, further comprising at least one of drying the nanoparticles or dispersing the nanoparticles in an aqueous solution.

5. The method of clause 2, wherein the suitable solvent comprises at least one of water, dichloromethane, acetonitrile, chloroform, dimethylformamide, tetrahydrofuran, ethanol, ethyl acetate, or acetone.

6. A method, for preparing polyoxalate nanoparticles for delivery of a drug, comprising:
(a) mixing 1,4-cyclohexanedimethanol (CDE) with oxalyl chloride (OC) in the presence of dichloromethane (DCM), and triethylamine to produce polyoxalate polymer;
(b) mixing the polyoxalate polymer with a drug in the presence of polyvinylalcohol and a suitable solvent to form a substantially homogeneous emulsion of polyoxalate nanoparticles containing the drug.

7. The method of clause 6, wherein the formed polyoxalate nanoparticles containing the drug have diameters ranging from about 50 to about 300 nm.

8. The method of clause 6, further comprising at least one of drying the nanoparticles containing the drug or dispersing the nanoparticles containing the drug in an aqueous solution.

9. The method of clause 6, further comprising solidifying the polyoxalate nanoparticles containing the drug at room temperature.

10. The method of clause 9, further comprising redispersing the solidified polymeric mixture in an aqueous solution.

11. The method of clause 6, wherein the suitable solvent comprises at least one of water, dichloromethane, acetonitrile, chloroform, dimethyl formamide, tetrahydrofuran, ethanol, ethyl acetate, or acetone.

12. Drug delivery nanoparticles comprising polyoxalate polymers, wherein the nanoparticles have diameters ranging from about 50 to about 300 nm.

13. The drug delivery nanoparticles of clause 12, wherein the nanoparticles comprise a payload.

14. The drug delivery nanoparticles of clause 13, wherein the payload comprises a therapeutic or diagnostic agent.

15. The drug delivery nanoparticles of clause 14, wherein the therapeutic agent is selected from the group consisting of methazolamide, insulin and cholecystokinin (CCK).

16. The drug delivery nanoparticles of clause 15, wherein the cholecystokinin is any one of cholecystokinin-8 (CCK-8), N-sarkosyl-CCK-8, N-taurine-CCK-8, N-pyroglutamic-CCK-8, C-terminal heptapeptide of CCK (CCK-7), N-sarkosyl-CCK-7, N-taurine-CCK-7, N-pyroglutamic-CCK-7, t-BOCK-CCK-7 or cholecystokinin-4 (CCK-4).

17. A composition comprising drug delivery nanoparticles comprising polyoxalate polymers, wherein the nanoparticles have diameters ranging from about 50 to about 300 nm.

18. The composition of clause 17, wherein the nanoparticles further comprise a payload.

19. The composition of clause 18, wherein the payload comprises a therapeutic or diagnostic agent.

20. The composition of clause 19, wherein the therapeutic agent is selected from the group consisting of methazolamide, insulin and cholecystokinin (CCK).

21. The composition of clause 20, wherein the cholecystokinin (CCK) is any one of cholecystokinin-8 (CCK-8), N-sarkosyl-CCK-8, N-taurine-CCK-8, N-pyroglutamic-CCK-8, C-terminal heptapeptide of CCK (CCK-7), N-sarkosyl-CCK-7, N-taurine-CCK-7, N-pyroglutamic-CCK-7, t-BOCK-CCK-7 or cholecystokinin-4 (CCK-4).

22. Drug delivery nanoparticles comprising polyoxalate polymers, wherein the nanoparticles have diameters ranging from about 50 to about 300 nm, and wherein the nanoparticles further comprise a functional moiety.

23. The drug delivery nanoparticles of clause of clause 22, wherein the functional moiety comprises 2-deoxyglucose.

Some embodiments of the subject technology provide biocompatible polymeric nanoparticles comprising polyoxalate polymers, which are nontoxic and safe to the body, with an average diameter of about 100-300 nm as measured by TEM (transmission electron microscopy).

Some embodiments include polyoxalate nanoparticles having diameters ranging from about 100 to about 300 nm.

Some embodiments include a method, for preparing biocompatible nanoparticles of polyoxalate polymers, comprising: (a) mixing 1,4-cyclohexanedimethanol (CDE) with oxalyl chloride (OC) in the presence of, dichloromethane (DCM, also know as methylene chloride), and triethylamine to produce polyoxalate polymer; and (b) adding the polyoxalate polymer to an aqueous solution of polyvinylalcohol (PVA) in the presence of a suitable solvent to produce a substantially homogenous emulsion of polyoxalate nanoparticles.

Some embodiments further include at least one of drying the nanoparticles or re-dispersing the nanoparticles in an aqueous solution.

Some embodiments include a method, for preparing polyoxalate nanoparticles for delivery of a drug, comprising: (a) mixing 1,4-cyclohexanedimethanol (CDE) with oxalyl chloride (OC) in the presence of dichloromethane (DCM, methylene chloride), and triethylamine to produce polyoxalate polymer; (b) mixing the polyoxalate polymer with the drug in the presence of polyvinylalcohol and a suitable solvent to form a substantially homogeneous emulsion of polyoxalate nanoparticles containing the drug; and (c) solidifying the polyoxalate nanoparticles containing the drug at room temperature. In an embodiment, the suitable solvent comprises water, dichloromethane, acetonitrile, chloroform, dimethylformamide, tetrahydrofuran, ethanol, ethyl acetate, acetone, and the like.

Some embodiments further include dispersing the solidified polymeric mixture in an aqueous solution.

Additional features and advantages of the subject technology will be set forth in the description below, and in part will be apparent from the description, or may be learned by practice of the subject technology. The advantages of the subject technology will be realized and attained by the structure particularly pointed out in the written description and claims hereof as well as the appended drawings.

It is to be understood that both the foregoing general description and the following detailed description are exemplary and explanatory and are intended to provide further explanation of the subject technology as claimed.

### BRIEF DESCRIPTION OF THE DRAWINGS

The accompanying drawings, which are included to provide further understanding of the subject technology and are incorporated in and constitute a part of this specification, illustrate aspects of the subject technology and together with the description serve to explain the principles of the subject technology.

FIG. 1 schematically depicts the condensation reaction of 1,4-cyclohexanedimethanol and oxalyl chloride to produce polyoxalate.

FIG. 2 is a schematic showing of the steps carried out to make polyoxalate nanoparticles by an oil-in-water emulsion.

FIG. 3 shows the nanoparticles of the subject technology being measured by dynamic light scattering method.

FIG. 4 is a schematic showing of the steps carried out to make polyoxalate nanoparticles by an oil-in-water emulsion with methazolamide.

FIG. 5 is a graph showing the plasma CCK-8 concentrations over time in mice orally administered with CCK-loaded and unloaded polyoxalate nanoparticles. The graph line with filled squares represents the plasma CCK-8 concentration profile in mice dosed with CCK-8-loaded nanoparticles. The graph line with open squares represents the plasma CCK-8 concentration profile in mice dosed with empty nanoparticles.

FIG. 6 is a schematic showing of the reaction steps for producing 2-deoxyglucose (2-DG)-functionalized polyoxalate nanoparticles.

### DETAILED DESCRIPTION

In the following detailed description, numerous specific details are set forth to provide a full understanding of the subject technology. It will be apparent, however, to one ordinarily skilled in the art that the subject technology may be practiced without some of these specific details. In other instances, well-known structures and techniques have not been shown in detail so as not to obscure the subject technology.

In describing and claiming the subject technology, the following terminology will be used in accordance with the definitions set out below.

A phrase such as “an aspect” does not imply that such aspect is essential to the subject technology or that such aspect applies to all configurations of the subject technology. A disclosure relating to an aspect may apply to all configurations, or one or more configurations. An aspect may provide one or more examples of the disclosure. A phrase such as “an aspect” may refer to one or more aspects and vice versa. A phrase such as “an embodiment” does not imply that such embodiment is essential to the subject technology or that such embodiment applies to all configurations of the subject technology. A disclosure relating to an embodiment may apply to all embodiments, or one or more embodiments. An embodiment may provide one or more examples of the disclosure. A phrase such “an embodiment” may refer to one or more embodiments and vice versa.

As used herein, the phrase “at least one of” preceding a series of items, with the term “and” or “or” to separate any of the items, modifies the list as a whole, rather than each member of the list (i.e., each item). The phrase “at least one of” does not require selection of at least one of each item listed; rather, the phrase allows a meaning that includes at least one of any one of the items, and/or at least one of any combination of the items, and/or at least one of each of the items. By way of example, the phrases “at least one of A, B, and C” or “at least one of A, B, or C” each refer to only A, only B, or only C; any combination of A, B, and C; and/or at least one of each of A, B, and C.

Furthermore, to the extent that the term “include,” “have,” or the like is used in the description or the claims, such term is intended to be inclusive in a manner similar to the term “comprise” as “comprise” is interpreted when employed as a transitional word in a claim.

The word “exemplary” is used herein to mean “serving as an example, instance, or illustration.” Any embodiment described herein as “exemplary” is not necessarily to be construed as preferred or advantageous over other embodiments.

A reference to an element in the singular is not intended to mean “one and only one” unless specifically stated, but rather “one or more.” Pronouns in the masculine (e.g., his) include the feminine and neuter gender (e.g., her and its) and vice versa. The term “some” refers to one or more.

As used herein, the terms “payload” or “drug,” are used interchangeably, and refer to a bioactive agent, an active agent, a therapeutic agent, or a diagnostic agent that can be encapsulated by or be incorporated in the polyoxalate nanoparticles of the subject technology. An active agent or therapeutic agent is generally accepted in the art to be any compound or substance which is used to treat any given disease or disorder; it can also be a pharmaceutically acceptable agent, or a pharmaceutically acceptable salt thereof, including small molecules, chemical compounds or polynucleotide such as DNA, RNA and the like, or biologics such as proteins or peptides. A diagnostic agent is typically a contrast agent, such as an x-ray contrast agent, or any other type of diagnostic material. The drug exists as a discrete, crystalline phase. The crystalline phase differs from a non-crystalline or amorphous phase which results from precipitation techniques, such as those described in U.S. Pat. No. 5,133,908, which is hereby incorporated by reference herein in its entirety.

The term “pharmaceutically acceptable” as used herein refers to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The term “pharmaceutically acceptable salts” as used herein refers to derivatives wherein the parent compound is modified by making acid or base salts thereof. Examples of pharmaceutically acceptable salts include, but are not limited to, mineral or organic acid salts of basic residues such as amines; alkali or organic salts of acidic residues such as carboxylic acids; and the like. The pharmaceutically acceptable salts include the conventional non-toxic salts or the quaternary ammonium salts of the parent compound formed, for example, from non-toxic inorganic or organic acids. For example, such conventional non-toxic salts include those derived from inorganic acids such as hydrochloric, hydrobromic, sulfuric, sulfamic, phosphoric, nitric, and the like; and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, sulfanilic, 2-acetoxybenzoic, fumaric, toluenesulfonic, methanesulfonic, ethane disulfonic, oxalic, isethionic, and the like.

The term “therapeutic” refers to the alleviation, prevention, or inhibition of any undesired signs or symptoms of a disease or condition, to any extent. Such undesired signs may include those that worsen the subject's overall feeling of well-being or appearance. This term does not necessarily indicate total cure or abolition of the disease or condition. A “therapeutic agent” is a compound that, upon administration to a mammal in a therapeutically effective amount, provides a therapeutic benefit to the mammal. A therapeutic agent may be referred to herein as a drug. Those skilled in the art will appreciate that the term “therapeutic agent” is not limited to drugs that have received regulatory approval. A “therapeutic agent” can be operatively associated with at least one carrier and/or other agent.

The terms “biocompatible polymer” and “biocompatibility” when used in relation to polymers are art-recognized. For example, biocompatible polymers include polymers that are neither themselves toxic to the host (e.g., an animal or human), nor degrade (if the polymer degrades) at a rate that produces monomeric or oligomeric subunits or other byproducts at toxic concentrations in the host. In certain embodiments of the subject technology, biodegradation generally involves degradation of the polymer in an organism, e.g., into its monomeric subunits, which may be known to be effectively non-toxic.

The term “poorly water soluble” as used herein refers to payloads having a solubility of less than about 30 mg/ml, less than about 20 mg/ml, less than about 10 mg/ml, or less than about 1 mg/ml. Such payloads tend to be eliminated from the gastrointestinal tract before being absorbed into the circulation.

A “patient,” “subject,” or “host” to be treated by the subject method may mean either a human or non-human animal, such as primates, mammals, and vertebrates.

The phrase “therapeutically effective amount” is an art-recognized term. In certain embodiments, the term refers to an amount of the therapeutic agent that, when incorporated into a polymer of the subject technology, produces some desired effect at a reasonable benefit/risk ratio applicable to any medical treatment. The effective amount may vary depending on such factors as the disease or condition being treated, the particular targeted constructs being administered, the size of the subject, or the severity of the disease or condition. One of ordinary skill in the art may empirically determine the effective amount of a particular compound without necessitating undue experimentation. The term “ED50” is art-recognized. In certain embodiments, ED50 means the dose of a drug that produces 50% of its maximum response or effect, or, alternatively, the dose that produces a pre-determined response in 50% of test subjects or preparations. The term “LD50” is art-recognized. In certain embodiments, LD50 means the dose of a drug that is lethal in 50% of test subjects. The term “therapeutic index” is an art-recognized term that refers to the therapeutic index of a drug, defined as LD50/ED50.

As used herein, the term “functional group,” used interchangeably with “functional moiety” and “functional ligand,” refers to a chemical group that imparts a particular function to a nanoparticle bearing the chemical group. For example, functional groups can include macromolecular substances such as antibodies, oligonucleotides, carbohydrates, biotin, or streptavidin, polypeptides (including polypeptides that comprise non-amino acid moieties such as phosphate groups, sugars, carbohydrates, lipids, etc.), and hormones. Functional groups can include macromolecular substances that are known to bind particular molecules, where such macromolecular substances are members of specific binding pairs. Functional groups can include small chemical groups comprising moieties such as amines, amides, pyridinium, quinazolines, heterocyclic groups, aryl groups, carboxylates, and the like. Functional groups can comprise a radioactive moiety. For example, a functional group includes any of the foregoing groups, where the group is radioactive.

The subject technology relates to a method for preparing biocompatible polymeric nanoparticles comprising polyoxalate. Some embodiments of the subjection technology relate to a method of preparing polyoxalate-based nanoparticles that can be used as drug delivery vehicles.

Some embodiments of the subject technology provide biocompatible polymeric nanoparticles comprising polyoxalate polymers, which are nontoxic and safe to the body, with an average diameter of about 100-300 nm as measured by TEM (transmission electron microscopy).

Some embodiments of the subject technology provide biocompatible polymeric nanoparticles comprising polyoxalate polymers, which are nontoxic and safe to the body, which can contain a high load of hydrophobic (water-insoluble or poorly water-soluble) drugs and release the drugs at controlled rates and/or at target sites in the body.

Some embodiments of the subject technology provide biocompatible polymeric nanoparticles comprising polyoxalate polymers, which are nontoxic and safe to the body, which can contain a, high load of hydrophilic (water-soluble) drugs and release the drugs at controlled rates and/or at target sites in the body.

Some embodiments of the subject technology provide biocompatible polymeric nanoparticles comprising polyoxalate polymers, which are nontoxic and safe to the body, which can contain a high load of hydrophobic or hydrophilic drugs or both and release the drugs at controlled rates and/or at target sites in the body.

Some embodiments of the subject technology provide a method for preparing biocompatible polymeric nanoparticle aggregates comprising polyoxalate polymers, which are nontoxic and safe to the body, which can contain a high load of hydrophobic (insoluble or poorly soluble) or hydrophilic (water-soluble) drugs or both and release the drugs at controlled rates or at target sites in the body.

Some embodiments of the subject technology provide a method for the preparation of biocompatible polymeric nanoparticles comprising polyoxalate polymers, which is safe for the environment and to the body.

In an embodiment, the subject technology provides a method for preparing biocompatible polymeric nanoparticles made up of polyoxalate polymers comprising: (1) mixing 1,4-cyclohexanedimethanol (CDE) with oxalyl chloride (OC) in the presence of dichloromethane (DCM, methelene chloride) and triethylamine to produce polyoxalate polymer; (2) adding the polyoxalate polymer to a solution of polyvinylalcohol (PVA) in the presence of methelene choloride to produce a homogenous emulsion of polyoxalate nanoparticles; and (3) drying said nanoparticels and/or optionally dispersing or re-dispersing the solidified nanoparticles in an aqueous solution.

In an aspect related to the above embodiment, the polyoxalate nanoparticles of the subject technology have an average diameter of about 100-300 nm as measured by TEM (transmission electron microscopy).

In another embodiment, the subject technology provides a method for preparing polyoxalate nanoparticles for drug delivery, comprising: mixing a polyoxalate polymer prepared in step (1) above with a drug in the presence of polyvinylalcohol and a suitable solvent (e.g., aqueous, organic or inorganic or mixture thereof) to form a homogeneous emulsion of polyoxalate nanoparticles containing the drug; solidifying the polyoxalate nanoparticles containing the drug at room temperature; and optionally dispersing or redispersing the solidified polymeric mixture in an aqueous solution.

In an aspect related to the above embodiment, the polyoxalate nanoparticles containing the drug (e.g., methazolamine, CCK-8, or fluorescent moiety such as 4-dimethylamino-4′-nitrostilbene) have an average diameter of about 100 to about 200 nm, or about 100 to 300 nm, as measured by dynamic light scattering method.

In an embodiment, the polyoxalate nanoparticles of the subject technology, with or without a drug payload, have a diameter ranging anywhere between about 50 to about 100 nm, or between about 100 to about 200 nm, or between about 200 to about 300 nm, or between about 300 to about 400 nm, or between about 400 to about 500 nm, or between about 500 to about 650 nm, or between about 650 to about 800 nm, or between about 800 to about 1000 nm.

In another embodiment, the subject technology pertains to a method for the preparation of biocompatible polymeric nanoparticle aggregates made up of polyoxalate for drug delivery, comprising (1) mixing 1,4-cyclohexanedimethanol (CDE) with pre-cooled oxalyl chloride (OC) in the presence of dichloromethane and triethylamine to produce polyoxalate polymer; (2) adding the polyoxalate polymer to a solution of polyvinylalcohol (PVA) in the presence of methelene choloride to produce a homogenous emulsion of polyoxalate nanoparticles; and (3) drying said nanoparticels and/or optionally dispersing or redispersing the solidified nanoparticles in an aqueous solution for use.

In another embodiment, the subject technology provides a method for preparing biocompatible polymeric nanoparticle aggregates made up of polyoxalate for drug delivery, comprising: mixing a polyoxalate polymer prepared in step (I) above with a drug in the presence of polyvinylalcohol and a suitable solvent (e.g., aqueous, organic or inorganic or mixture thereof) to form a homogeneous emulsion of polyoxalate nanoparticles containing the drug; solidifying the polyoxalate nanoparticles containing the drug at room temperature; and optionally dispersing or redispersing the solidified polymeric mixture in an aqueous solution for use.

A. Method of Preparation of the Polyoxalate Nanoparticles of the Subject Technology

The preparation of biocompatible polymeric nanoparticles for drug delivery in accordance with the subject technology is useful for easily producing polyoxalate nanoparticles at low cost. The poloxalate nanoparticles prepared using the method of the subject technology show desired particle sizes suitable for use in drug delivery and a uniform particle size distribution. The polyoxalate nanoparticles of the subject technology can contain water-soluble, poorly water-soluble or insoluble drugs. Also, the polyxalate nanoparticles are nontoxic, biocompatible, and contain no organic solvents and are thus safe for use in the body. Further, after being administered in the body, the polyoxalate nanoparticles of the subject technology, with a high content of water-soluble, poorly water-soluble, or insoluble drug entrapped therein, can safely deliver the drug to target sites in the body or can stably release the drug at a controlled rate.

The polyoxalate nanoparticles of the subject technology can be made by (1) mixing 1,4-cyclohexanedimethanol (CDE) with oxalyl chloride (OC) in the presence of dichlorornethane (DCM, methelene chloride) and triethylamine to produce polyoxalate polymer; (2) adding the polyoxalate polymer to a solution of polyvinylalcohol (PVA) in the presence of methelene choloride to produce a homogenous emulsion of polyoxalate nanoparticles; and (3) drying said nanoparticels and/or optionally dispersing or re-dispersing the solidified nanoparticles in an aqueous solution.

Alternatively or in addition, the polyoxalate nanoparticles of the subject technology, comprising a payload (e.g., drug, therapeutic agent, diagnostic agent, bioactive agent, active agent) can be made by mixing a polyoxalate polymer prepared in step (1) above with the payload in the presence of polyvinylalcohol and a suitable solvent (e.g., aqueous, organic or inorganic or mixture thereof) to form a homogeneous emulsion of polyoxalate nanoparticles containing the payload; optionally dispersing the formed polyoxalate nanoparticles containing the payload or solidifying them at room temperature. In a related aspect, the method also includes redispersing the solidified polymeric mixture in an aqueous solution.

In an embodiment, the polyoxalate nanoparticles of the subject technology, with or without a drug payload, have a diameter ranging anywhere between about 50 to about 1000 nm. More particularly, these nanoparticles have a diameter ranging between about 50 to about 100 nm, or between about 100 to about 200 nm, or between about 200 to about 300 nm, or between about 300 to about 400 nm, or between about 400 to about 500 nm, or between about 500 to about 650 nm, or between about 650 to about 800 nm, or between about 800 to about 1000 nm.

B. Dispersibility or Dispersibility Profiles of the Polyoxalate Nanoparticles of the Subject Technology

An additional feature of the nanoparticles of the subject technology is that the polyoxalate nanoparticles, with or without a drug or payload, can disperse (upon preparation) or redisperse (after having been dried or solidified) such that the effective average particle size of the dispersed or redispersed nanoparticles is less than about 800 nm, or less than about 500 nm, or preferably less than about 300 nm. This is significant, because the nanoparticles of the subject technology benefit from the small particle size; if drug-loaded polyoxlate nanoparticles do not disperse or redisperse into the small particle sizes, upon administration, they can form clumps or agglomerates, which results in a reduced bioavailability of the dosage form.

In an embodiment, prior to administration of the drug-loaded polyoxalate nanoparticles of the subject technology, the nanoparticles are dispersed (if they were just prepared) or redispersed (if they were previously solidified) in a biorelevant aqueous media. Such biorelevant aqueous media can be any aqueous media that exhibit the desired ionic strength and pH, which form the basis for the biorelevance of the media. The desired pH and ionic strength are those that are representative of physiological conditions found in the human body. Such biorelevant aqueous media can be, for example, aqueous electrolyte solutions or aqueous solutions of any salt, acid, or base, or a combination thereof, which exhibit the desired pH and ionic strength.

Biorelevant pH is well known in the art. For example, in the stomach, the pH ranges from slightly less than 2 (but typically greater than 1) up to 4 or 5. In the small intestine the pH can range from 4 to 6, and in the colon it can range from 6 to 8. Biorelevant ionic strength is also well known in the art. Fasted state gastric fluid has an ionic strength of about 0.1 M while fasted state intestinal fluid has an ionic strength of about 0.14. It is believed that the pH and ionic strength of the biorelevant media is more critical than the specific chemical content. Accordingly, appropriate pH and ionic strength values can be obtained through numerous combinations of strong acids, strong bases, salts, single or multiple conjugate acid-base pairs (i.e., weak acids and corresponding salts of that acid), monoprotic and polyprotic electrolytes, etc.

Representative electrolyte solutions can be, but are not limited to, HCl solutions, ranging in concentration from about 0.001 to about 0.1 M, and NaCl solutions, ranging in concentration from about 0.001 to about 0.1 M, and mixtures thereof. For example, electrolyte solutions can be, but are not limited to, about 0.1 M HCl or less, about 0.01 M HCl or less, about 0.001 M or less, about 0.1 M NaCl or less, about 0.01 M NaCl or less, about 0.001 M NaCl or less, and mixtures thereof. Of these electrolyte solutions, 0.01 M HCl and/or 0.1 M NaCl, are most representative of fasted human physiological conditions, owing to the pH and ionic strength conditions of the proximal gastrointestinal tract.

Electrolyte concentrations of 0.001 M HCl, 0.01 M HCl, and 0.1 M HCl correspond to pH 3, pH 2, and pH 1, respectively. Thus, a 0.01 M HCl solution simulates typical acidic conditions found in the stomach. A solution of 0.1 M NaCl provides a reasonable approximation of the ionic strength conditions found throughout the body, including the gastrointestinal fluids, although concentrations higher than 0.1 M may be employed to simulate fed conditions within the human GI tract.

Exemplary solutions of salts, acids, bases or combinations thereof, which exhibit the desired pH and ionic strength, include but are not limited to phosphoric acid/phosphate salts+sodium, potassium and calcium salts of chloride, acetic acid/acetate salts+sodium, potassium and calcium salts of chloride, carbonic acid/bicarbonate salts+sodium, potassium and calcium salts of chloride, and citric acid/citrate salts+sodium, potassium and calcium salts of chloride.

In some embodiments of the subject technology, the dispersed or redispersed drug-loaded polyoxalate nanoparticles (in an aqueous, biorelevant, or any other suitable media) have an effective average particle size of less than about 1900 nm, less than about 1800 nm, less than about 1700 nm, less than about 1600 nm, less than about 1500 nm, less than about 1400 nm, less than about 1300 nm, less than about 1200 nm, less than about 1100 nm, less than about 1000 nm, less than about 900 nm, less than about 800 nm, less than about 700 nm, less than about 600 nm, less than about 500 nm, less than about 400 nm, less than about 300 nm, less than about 250 nm, less than about 200 nm, less than about 150 nm, less than about 100 nm, less than about 75 mm, or less than about 50 nm, as measured by light-scattering methods, microscopy, or other appropriate methods.

Redispersibility can be tested using any suitable means known in the art. See e.g., the example sections of U.S. Pat. No. 6,375,986 for “Solid Dose Nanoparticulate Compositions Comprising a Synergistic Combination of a Polymeric Surface Stabilizer and Dioctyl Sodium Sulfosuccinate,” which is hereby incorporated by reference herein in its entirety.

C. Drugs or Payloads of Polyoxalate Nanoparticles of the Subject Technology

The nanoparticles of the subject technology comprise a therapeutic or diagnostic agent, which are collectively referred to as a “drug” or “payload.” A therapeutic agent can be a pharmaceutical, including biologics such as proteins and peptides, and a diagnostic agent is typically a contrast agent, such as an x-ray contrast agent, or any other type of diagnostic material. The drug exists as a discrete, crystalline phase. The crystalline phase differs from a non-crystalline or amorphous phase which results from precipitation techniques, such as those described in U.S. Pat. No. 5,133,908, which is hereby incorporated by reference herein in its entirety.

The subject technology can be practiced with a wide variety of drugs. Exemplary drugs of the subject technology include, but are not limited to methazolamide, insulin; growth factors, such as epidermal growth factor (EGF), insulin-like growth factor (IGF), transforming growth factor (TGF), nerve growth factor (NGF), platelet-derived growth factor (PDGF), bone morphogenic protein (BMP), fibroblast growth factor and the like; somatostatin; somatotropin; somatropin; somatrem; calcitonin; parathyroid hormone; colony stimulating factors (CSF); clotting factors; tumor necrosis factors: interferons; interleukins; gastrointestinal peptides, such as vasoactive intestinal peptide (VIP), cholecystokinin (CCK), gastrin, secretin, and the like; erythropoietins; growth hormone and GRF; vasopressins; octreotide; pancreatic enzymes; dismutases such as superoxide dismutase; thyrotropin releasing hormone (TRH); thyroid stimulating hormone; luteinizing hormone; LHRH; GHRH; tissue plasminogen activators; macrophage activator; chorionic gonadotropin; heparin; atrial natriuretic peptide; hemoglobin; retroviral vectors; relaxin; cyclosporin; oxytocin; vaccines; monoclonal antibodies; and the like; and analogs and derivatives of these compounds.

The drug can be in an essentially pure form, soluble, poorly soluble or insoluble in water. By “poorly water-soluble” it is meant that the drug has a solubility in water of less than about 10 mg/mL, and preferably of less than about 1 mg/mL.

The drugs that can be used with the polyoxalate nanoparticles of the subject technology can be selected from a variety of known classes of drugs, including, for example, analgesics, anti-inflammatory agents, anthelmintics, anti-arrhythmic agents, antibiotics (including penicillins), anticoagulants, antidepressants, antidiabetic agents, antiepileptics, antihistamines, antihypertensive agents, antimuscarinic agents, antimycobacterial agents, anti neoplastic agents, immunosuppressants, antithyroid agents, antiviral agents, anxiolytic sedatives (hypnotics and neuroleptics), astringents, beta-adrenoceptor blocking agents, blood products and substitutes, cardiac inotropic agents, contrast media, corticosteroids, cough suppressants (expectorants and mucolytics), diagnostic agents, diagnostic imaging agents, diuretics, dopaminergics (antiparkinsonian agents), haemostatics, immunological agents, lipid regulating agents, muscle relaxants, parasympathomimetics, parathyroid calcitonin and biphosphonates, prostaglandins, radio-pharmaceuticals, sex hormones (including steroids), anti-allergic agents, stimulants and anoretics, sympathomimetics, thyroid agents, vasodilators and xanthines.

A description of these classes of drugs and a listing of species within each class can be found in the Physicians' Desk Reference® (Medical Economies Company, Inc. 2011), incorporated herein by reference. Contrast agents are taught in the U.S. Pat. No. 5,145,684, which is specifically incorporated by reference. Suitable diagnostic agents are also disclosed in U.S. Pat. No. 5,260,478; U.S. Pat. No. 5,264,610; U.S. Pat. No. 5,322,679; all specifically incorporated herein by reference.

In an embodiment, the subject technology relates to methazolamide-loaded nanoparticles comprising a polyoxalate matrix. In an embodiment, the subject technology relates to cholecystokinin (CCK)-loaded nanoparticles comprising a polyoxalate matrix. In an embodiment, the polyoxalate nanoparticles of the subject technology can adhere to the intestinal lining and gradually pass through the lining into the circulatory system, where they gradually release their payload of a drug or bioactive agent at a rate determined by the decomposition rate of the core. In another embodiment, the drug-loaded nanoparticles of the subject technology are capable of avoiding first pass hepatic metabolism, which occurs when the drugs are removed from the bloodstream as they pass through the liver.

Cholecystokinin (CCK), its derivatives, analogs and variants are known for their appetite suppressing activities. Fragments and derivatives of a CCK of particular interest include without limitation cholecystokinin-8 (CCK-8), N-sarkosyl-CCK-8, N-taurine-CCK-8, N-pyroglutamic-CCK-8, C-terminal heptapeptide of CCK (CCK-7), N-sarkosyl-CCK-7, N-taurine-CCK-7, N-pyroglutamic-CCK-7, t-130CK-CCK-7, and cholecystokinin-4 (CCK-4). The sulfated form of CCK-8 has a high affinity for the CCKA receptors, while the non-sulfated form of CCK-8, as well as CCK-4, gastrin, and pentagastrin (CCK-5) have a 10,000 fold lower affinity for these receptors (see, de Montigny, 1989, Arch. Gen. Psychiatry 46(6): 511, which is incorporated by reference herein in its entirety). The CCKB receptors exhibit a high affinity and selectivity for CCK-4, gastrin, pentagastrin (CCK-5), and the non-sulfated CCK-8. Sulfated CCK-8 has a slightly lower or same affinity for CCKB receptors (see, de Montigny, 1989, Arch. Gen. Psychiatry 46(6): 511; Bradwcjn et al., 1992b, Am. J. Psychiatry 149: 962, each of which is incorporated by reference herein in its entirety). Thus, sulfated CCK-8 is preferred in certain embodiments. Other appetite suppressing moieties contemplated include any CCKA agonist having appetite suppressing properties, caerulein, Bombesin, and all other fragments of CCK containing at least the four C-terminal amino acids (Trp-Met-Asp-Phe-NH₂, SEQ ID NO:1) (see, Abhiram, 2004, Endocrinology 145: 2613, which is incorporated by reference herein in its entirety).

CCK was first identified in 1928 from preparations of intestinal extracts by its ability to stimulate gallbladder contraction. Other biological actions of CCK have since been reported, including stimulation of pancreatic secretion, delayed gastric emptying, stimulation of intestinal motility and stimulation of insulin secretion. The actions of CCK, also reportedly include effects on cardiovascular function, respiratory function, neurotoxicity and seizures, cancer cell proliferation, analgesia, sleep, sexual and reproductive behaviors, memory, anxiety and dopamine-mediated behaviors (Crawley and Corwin, 1994, Peptides 15: 731, which is incorporated by reference herein in its entirety). Other reported effects of CCK include stimulation of pancreatic growth, stimulation of gallbladder contraction, inhibition of gastric acid secretion, pancreatic polypeptide release and a contractile component of peristalsis. Additional reported effects of CCK include vasodilation.

It has been reported that injections of combinations of glucagon, CCK and bombesin potentiated the inhibition of intake of condensed milk test meals in non-deprived rats over the inhibitions observed with individual compounds. It has also been reported that glucagon and CCK synergistically inhibit sham feeding in rats. It has also been suggested that estradiol and CCK can have a synergistic effect on satiety. Experimental manipulations of exogenous and endogenous CCK and estradiol have produced converging evidence that estradiol cyclically increases the activity of the CCK satiation-signaling pathway so that meal size and food intake decrease during the ovulatory or estrous phase in animals. It is common-place for women who begin oral administration of estrogen (hormone replacement therapy or birth control) to gain weight. This occurs through several mechanisms including water retention; increased production of sex hormone binding globulin which thereby decreases bio-available testosterone; decrease in the production of IGF1. Thus, if oral estrogen replacement were given concurrently with an orally bio-available form of CCK, as proposed herein, it is likely that the usual weight gain would not occur. This would certainly be advantageous to women prone to obesity. It has also been proposed that signals arising from the small intestine in response to nutrients therein may interact synergistically with CCK to reduce food intake.

Additionally, it has been reported that CCK induces satiety in several species. For example, it has been reported that feeding depression was caused by CCK injected intra-peritoneally in rats, intra-arterially in pigs, intravenously in cats and pigs, into the cerebral ventricles in monkeys, rats, dogs and sheep, and intravenously in obese and nonobese humans. Studies from several laboratories have reportedly confirmed the behavioral specificity of low doses of CCK on inhibition in feeding, by comparing responding for food to responding for nonfood reinforcers in both monkeys and rats and by showing that CCK elicits the sequence of behaviors normally observed after meal ingestion (i.e., the postprandial satiety sequence). Additionally, comparison of behavior after CCK to behavior after food ingestion, alone or in combination with CCK has reportedly revealed behavioral similarities between CCK and food ingestion (see e.g., Crawley and Corwin, supra). It has also been reported that CCK in physiological plasma concentrations inhibits food intake and increases satiety in both lean and obese humans.

CCK was characterized in 1966 as a 33-amino acid peptide (Crawley and Corwin, supra). Species-specific molecular variants of the amino acid sequence of CCK have been identified. The 33-amino acid sequence and a truncated peptide, its 8-amino acid C-terminal sequence (CCK-8) have been reportedly identified in pig, rat, chicken, chinchilla, dog and humans. A 39-amino acid sequence was reportedly found in pig, dog and guinea pig. A 58-amino acid sequence was reported to have been found in cat, dog and humans. Frog and turtle reportedly show 47-amino acid sequences homologous to both CCK and gastrin. Very fresh human intestine has been reported to contain small amounts of an even larger molecule, termed CCK-83. In the rat, a principal intermediate form has been reportedly identified, and is termed CCK-22 (Physiology of the Gastrointestinal Tract, 3d Ed., Walsh, 1994; Raven Press, New York, N.Y. 1994, which is incorporated by reference herein in its entirety).

A nonsulfated CCK-8 and a tetrapeptide (termed CCK-4 (CCK30-33)) have been reported in rat brain. The C-terminal penta peptide (termed CCK-4 (CCK 29-33)) conserves the structural homology of CCK, and homology with the neuropeptide, gastrin. The C-terminal sulfated octapeptide sequence, CCK-8, Asp-Tyr(SO3H)-Met-Gly-Trp-Met-Asp-Phe-NH2 (SEQ ID NO:2), is reportedly relatively conserved across species. Cloning and sequence analysis of a cDNA encoding preprocholecystokinin from rat thyroid carcinoma, porcine brain, and porcine intestine reportedly revealed 345 nucleotides coding for a precursor to CCK, which is 115 amino acids and contains all of the CCK sequences previously reported to have been isolated (see, Crawley and Corwin, supra).

CCK is said to be distributed throughout the central nervous system and in endocrine cells and enteric nerves of the upper small intestine. CCK agonists include CCK itself (also referred to as CCK-33), CCK-8 (CCK26-33), non-sulfated CCK-8, pentagastrin (CCK-5 or CCK(29-33)), and the tetrapeptide, CCK-4 (CCK30-33). At the pancreatic CCK receptor, CCK-8 reportedly displaced binding with a 1000-5000 greater potency than unsulfated CCK-8 or CCK-4, and CCK-8 has been reported to be approximately 1000-fold more potent than unsulfated CCK-8 or CCK-4 in stimulating pancreatic amylase secretion (see, Crawley and Corwin, supra). In homogenates from the cerebral cortex, CCK receptor binding was said to be displaced by unsulfated CCK-8 and by CCK-4 at concentrations that were equimolar, 10-fold or 100-fold greater than sulfated CCK-8.

Receptors for CCK have been reportedly identified in a variety of tissues, and two primary subtypes have been described: type A receptors and type B receptors. Type A receptors have been reported in peripheral tissues including pancreas, gallbladder, pyloric sphincter and afferent vagal fibers, and in discrete areas of the brain. The type A receptor subtype (CCKA) has been reported to be selective for the sulfated octapeptide. Accordingly, in certain embodiments of the disclosure, the CCK fragment includes at least one sulfation group. CCKA agonists also include A-71623 and A-708874, which were developed based on the structure of CCK-4. Members of another series of CCKA agonists, which includes JMV-180, are reportedly active in stimulating pancreatic amylase release and inhibiting feeding (Crawley and Corwin, supra). Examples of non-peptide CCKA agonists are L-364718 and FPL 15849KF (Crawley and Corwin, supra and Morley et al., 1994, Am. J. Physiol. 267: R178, which is incorporated by reference herein in its entirety). Accordingly, substances which function as Type-A receptor-selective CCK agonists which may serve as anorectic agents are contemplated appetite suppressing moieties. These may include, without limitation, cholecystokinin-8 (CCK-8), N-sarkosyl-CCK-8, N-taurine-CCK-8, N-pyroglutamic-CCK-8, C-terminal heptapeptide of CCK (CCK-7), N-sarkosyl-CCK-7, N-taurine-CCK-7, N-pyroglutamic-CCK-7, t-BOCK-CCK-7, cholecystokinin-4 (CCK-4), caerulein, Bombesin, and all other fragments of CCK containing at least the four C-terminal amino acids (Trp-Met-Asp-Phc-NH2, SEQ ID NO:1).

D. Pharmacokinetic Profiles of the Drug-loaded Polyoxalate Nanoparticles of the Subject Technology

In an embodiment, when the polyoxalate nanoparticles of the subject technology, including a drug or drugs, are administered into the body, the polymeric components begin to degrade, thereby releasing the drug(s) at a controlled rate and/or at a target sites in the body. In an aspect, after being administered into the body, the nanoparticles can safely reach a target site with the drug entrapped within the nanoparticles.

In another embodiment, the nanoparticles of the subject technology increase the absorption of their drug payload across the blood brain barrier and/or the gastrointestinal barrier.

The subject technology also provides drug-loaded polyoxalate nanoparticles compositions having a desirable pharmacokinetic profile when administered to mammalian subjects. The desirable pharmacokinetic profile of drug-loaded polyoxalate nanoparticles compositions preferably includes, but is not limited to: (1) a Tₘₐₓ for an active agent, when assayed in the plasma of a mammalian subject following administration, that is preferably less than the Tₘₐₓ for a composition of the same active agent at the same dosage but without being incorporated in polyoxalate nanoparticles; (2) a Cₘₐₓ for an active agent, when assayed in the plasma of a mammalian subject following administration, that is preferably greater than the Cₘₐₓ for a composition of the same active agent at the same dosage but without being incorporated in polyoxalate nanoparticles; and/or (3) an area under the curve (AUC) for an active agent, when assayed in the plasma of a mammalian subject following administration, that is preferably greater than the AUC for a composition of the same active agent at the same dosage but without being incorporated in polyoxalate nanoparticles. AUC is generally known as the area under the plot of plasma concentration of drug (not logarithm of the concentration) against time after drug administration.

The desirable pharmacokinetic profile, as used herein, is the pharmacokinetic profile measured after the initial dose of the drug-loaded polyoxalate nanoparticles. The compositions can be formulated in any way as described herein and as known to those of skill in the art.

In an embodiment, a drug-loaded polyoxalate nanoparticles composition of the subject technology exhibits, in comparative pharmacokinetic testing with a composition of the same drug at the same dosage but without being incorporated in polyoxalate nanoparticles, a Tₘₐₓ not greater than about 100%, not greater than about 90%, not greater than about 80%, not greater than about 70%, not greater than about 60%, not greater than about 50%, not greater than about 40%, not greater than about 30%, not greater than about 25%, not greater than about 20%, not greater than about 15%, not greater than about 10%, or not greater than about 5% of the Tₘₐₓ exhibited by the composition of the same drug at the same dosage but without being incorporated in polyoxalate nanoparticles. This shorter Tₘₐₓ translates into a faster onset of therapeutic activity.

In another embodiment, a drug-loaded polyoxalate nanoparticles composition of the subject technology exhibits, in comparative pharmacokinetic testing with a-composition of the same drug at the same dosage but without being incorporated in polyoxalate nanoparticles, a Cₘₐₓ which is at least about 10%, at least about 20%, at least about 30%, at least about 40%, at least about 50%, at least about 60%, at least about 70%, at least about 80%, at least about 90%, at least about 100%, at least about 110%, at least about 120%, at least about 130%, at least about 140%, at least about 150%, at least about 160%, at least about 170%, at least about 180%, at least about 190%, or at least about 200% greater than the Cₘₐₓ exhibited by the composition of the same drug at the same dosage but without being incorporated in polyoxalate nanoparticles.

In another embodiment, a drug-loaded polyoxalate nanoparticles composition of the subject technology exhibits, in comparative pharmacokinetic testing with a composition of the same drug at the same dosage but without being incorporated in polyoxalate nanoparticles, an AUC which is at least about 10%, at least about 20%, at least about 30%, at least about 40%, at least about 50%, at least about 60%, at least about 70%, at least about 80%, at least about 90%, at least about 100%, at least about 110%, at least about 120%, at least about 130%, at least about 140%, at least about 150%, at least about 160%, at least about 170%, at least about 180%, at least about 190%, or at least about 200% greater than the AUC exhibited by the composition of the same drug at the same dosage but without being incorporated in polyoxalate nanoparticles.

Any formulation giving the desired pharmacokinetic profile is suitable for administration according to the present methods.

The subject technology encompasses drug-loaded polyoxalate nanoparticles compositions wherein preferably the pharmacokinetic profile of the drug is not substantially affected by the fed or fasted state of a subject ingesting the composition. This means that there is no substantial difference in the quantity of drug absorbed or the rate of drug absorption when the drug-loaded polyoxalate nanoparticles compositions are administered in the fed versus the fasted state. Thus, the drug-loaded polyoxalate nanoparticles compositions of the subject technology can preferably substantially eliminate the effect of food on the pharmacokinetics of the active agent.

In another embodiment of the drug-loaded polyoxalate nanoparticles compositions of the subject technology when administered to a mammal in a fasted state, is bioequivalent to the pharmacokinetic profile of the same composition administered at the same dosage, when administered to a mammal in a fed state.

“Bioequivalency” is generally established by a 90% Confidence Interval (CI) of between 0.80 and 1.25 for both Cₘₐₓ and AUC under U.S. Food and Drug Administration (USFDA) regulatory guidelines, or a 90% Cl for AUC of between 0.80 to 1.25 and a 90% Cl for Cₘₐₓ of between 0.70 to 1.43 under the European Medicines Evaluation Agency (EMEA) regulatory guidelines (Tₘₐₓ is not relevant for bioequivalency determinations under USFDA and EMEA regulatory guidelines).

E. Compositions/Formulations of Polyoxalate Nanoparticles of the Subject Technology

In an embodiment, the subject technology provides a composition comprising polyoxalate nanoparticles, wherein the nanoparticles have diameters ranging from about 50 to about 300 nm.

In another embodiment, the subject technology relates to compositions, pharmaceutical compositions and/or formulations including polyoxalate nanoparticles containing a drug (or drugs) to be delivered and, optionally, an appropriate carrier. The drug containing nanoparticles of the subject technology can be delivered alone or in combination with excipients, or on, in, or blended with a polymer, preferably a mucoadhesive polymer. The formulation may be in the form of a liquid such as a dispersion or suspension of nanoparticles, or may be in a solid dosage form, such as tablets, capsules, multiparticulate formulations, beads, granules, or particles. The formulation may contain an enteric or non-enteric coating. Preferably the formulation is an oral dosage, formulation. Optionally, the polyoxalate nanoparticles may be blended with a polymer and/or excipients.

The compositions, pharmaceutical compositions formulations described herein may be administered to the subject by any suitable means. Non-limiting examples of methods of administration include, among others, (a) administration though oral pathways, which administration includes administration in capsule, tablet, granule, spray, syrup, or other such forms; (b) administration through non-oral pathways such as rectal, vaginal, intraurethral, intraocular, intranasal, or intraauricular, which administration includes administration as an aqueous suspension, an oily preparation or the like or as a drip, spray, suppository, salve, ointment or the like; (c) administration via injection, subcutaneously, intraperitoneally, intravenously, intramuscularly, intradermally, intraorbitally, intracapsularly, intraspinally, intrasternally, or the like, including infusion pump delivery; (d) administration locally such as by injection directly in the renal or cardiac area, e.g., by depot implantation; as well as (e) administration topically; as deemed appropriate by those of skill in the art for bringing the active compound into contact with living tissue.

F. Polyoxalate Nanoparticles Particle Size

The compositions and/or formulations of the subject technology contain drug-loaded polyoxalata nanoparticles which have an effective average particle size of less than about 1000 nm (i.e., 1 micron). In other embodiments of the subject technology, the drug-loaded nanoparticles have a size of less than about 1000 nm, less than about 900 nm, less than about 800 nm, less than about 700 nm, less than about 600 nm, less than about 500 nm, less than about 400 nm, less than about 300 nm, less than about 250 nm, less than about 200 nm, less than about 150 nm, less than about 100 nm, less than about 75 nm, or less than about 50 nm, as Measured by light-scattering methods, microscopy, or other appropriate methods.

By “an effective average particle size of less than about 1000 nm” it is meant that at least 50% by weight of the drug-loaded polyoxalata nanoparticles have a particle size less than the effective average, i.e., less than about 1000 nm, 900 nm, 800 nm, 500 nm, 300 nm, etc., when measured by the above-noted techniques. In other embodiments of the subject technology, at least about 70%, at least about 90%, at least about 95%, or at least about 99% of the drug-loaded polyoxalata nanoparticles have a particle size less than the effective average, i.e., less than about 1000 nm, 900 nm, 800 nm, 500 nm, 300 nm, etc.

G. Concentrations and/or Doses of Drugs in Polyoxalate Nanoparticles

The relative amounts of drugs in polyoxalate of the subject technology can vary widely. The optimal amount of the individual components can depend, for example, upon the particular drug selected, the hydrophilic lipophilic balance (HLB), melting point, and etc.

The concentration of the drug payload can vary from about 99.5% to about 0.001%, from about 95% to about 0.1%, or from about 90% to about 0.5%, by weight, based on the total combined dry weight of the polyoxalate nanoparticles and the drug.

The dose for the compositions or formulations of the subject technology can be tailored to achieve a desired effect, but will depend on such factors as weight, diet, concurrent medication and other factors which those skilled in the medical arts will recognize. More specifically, a therapeutically effective amount means an amount of compound effective to prevent, alleviate or ameliorate symptoms of disease or prolong the survival of the subject being treated. Determination of a therapeutically effective amount is well within the capability of those skilled in the art, especially in light of the detailed disclosure provided herein.

As will be readily apparent to one skilled in the art, the useful in vivo dosage to be administered and the particular mode of administration will vary depending upon the age, weight and mammalian species treated, the particular compounds employed, and the specific use for which these compounds are employed. The determination of effective dosage levels, that is the dosage levels necessary to achieve the desired result, can be accomplished by one skilled in the art using routine pharmacological methods. Typically, human clinical applications of products are commenced at lower dosage levels, with dosage level being increased until the desired effect is achieved. Alternatively, acceptable in vitro studies can be used to establish useful doses and routes of administration of the compositions identified by the present methods using established pharmacological methods.

In non-human animal studies, applications of potential products are commenced at higher dosage levels, with dosage being decreased until the desired effect is no longer achieved or adverse side effects disappear. The dosage may range broadly, depending upon the desired effects and the therapeutic indication. Typically, dosages may be about 10 microgram/kg to about 100 mg/kg body weight, preferably about 100 microgram/kg to about 10 mg/kg body weight. Alternatively dosages may be based and calculated upon the surface area of the patient, as understood by those of skill in the art.

The exact formulation, route of administration and dosage for the pharmaceutical compositions of the subject technology can be chosen by the individual physician in view of the patient's condition. Typically, the dose range of the composition administered to the patient can be from about 0.5 to about 1000 mg/kg of the patient's body weight. The dosage may be a single one or a series of two or more given in the course of one or more days, as is needed by the patient. In instances where human dosages for compounds have been established for at least some condition, the subject technology will use those same dosages, or dosages that are about 0.1% to about 500%, more preferably about 25% to about 250% of the established human dosage. Where no human dosage is established, as will be the case for newly-discovered pharmaceutical compositions, a suitable human dosage can be inferred from ED₅₀ or ID₅₀ values, or other appropriate values derived from in vitro or in vivo studies, as qualified by toxicity studies and efficacy studies in animals.

It should be noted that the attending physician would know how to and when to terminate, interrupt, or adjust administration due to toxicity or organ dysfunctions. Conversely, the attending physician would also know to adjust treatment to higher levels if the clinical response were not adequate (precluding toxicity). The magnitude of an administrated dose in the management of the disorder of interest will vary with the severity of the condition to be treated and to the route of administration. The severity of the condition may, for example, be evaluated, in part, by standard prognostic evaluation methods. Further, the dose and perhaps dose frequency, will also vary according to the age, body weight, and response of the individual patient. A program comparable to that discussed above may be used in veterinary medicine.

Although the exact dosage will be determined on a drug-by-drug basis, in most cases, some generalizations regarding the dosage can be made. The daily dosage regimen for an adult human patient may be, for example, an oral dose of about 0.1 mg to 2000 mg of each active ingredient, preferably about 1 mg to about 500 mg, e.g. 5 to 200 mg. In other embodiments, an intravenous, subcutaneous, or intramuscular dose of each active ingredient of about 0.01 mg to about 100 mg, preferably about 0.1 mg to about 60 mg, e.g. about 1 to about 40 mg is used. In cases of administration of a pharmaceutically acceptable salt, dosages may be calculated as the free base. In some embodiments, the composition is administered 1 to 4 times per day. Alternatively the compositions of the subject technology may be administered by continuous intravenous infusion, preferably at a dose of each active ingredient up to about 1000 mg per day. As will be understood by those of skill in the art, in certain situations it may be necessary to administer the compounds disclosed herein in amounts that exceed, or even far exceed, the above-stated, preferred dosage range in order to effectively and aggressively treat particularly aggressive diseases or infections. In some embodiments, the compounds will be administered for a period of continuous therapy, for example for a week or more, or for months or years.

Dosage amount and interval may be adjusted individually to provide plasma levels of the active moiety which are sufficient to maintain the modulating effects, or minimal effective concentration (MEC). The MEC will vary for each compound but can be estimated from in vitro data. Dosages necessary to achieve the MEC will depend on individual characteristics and route of administration. However, HPLC assays or bioassays can be used to determine plasma concentrations.

Dosage intervals can also be determined using MEC value. Compositions should be administered using a regimen which maintains plasma levels above the MEC for 10-90% of the time, preferably between 30-90% and most preferably between 50-90%.

In cases of local administration or selective uptake, the effective local concentration of the drug may not be related to plasma concentration.

The amount of composition administered may be dependent on the subject being treated, on the subject's weight, the severity of the affliction, the manner of administration and the judgment of the prescribing physician.

Compositions disclosed herein (e.g., the therapeutic composition that comprises drug-loaded polyoxalate nanoparticles) can be evaluated for efficacy and toxicity using known methods. For example, the toxicology of a particular compound, or of a subset of the compounds, sharing certain chemical moieties, may be established by determining in vitro toxicity towards a cell line, such as a mammalian, and preferably human, cell line. The results of such studies are often predictive of toxicity in animals, such as mammals, or more specifically, humans. Alternatively, the toxicity of particular compounds in an animal model, such as mice, rats, rabbits, or monkeys, may be determined using known methods. The efficacy of a particular compound may be established using several recognized methods, such as in vitro methods, animal models, or human clinical trials. Recognized in vitro models exist for nearly every class of condition, including but not limited to cancer, cardiovascular disease, and various immune dysfunction. Similarly, acceptable animal models may be used to establish efficacy of chemicals to treat such conditions. When selecting a model to determine efficacy, the skilled artisan can be guided by the state of the art to choose an appropriate model, dose, and route of administration, and regime. Of course, human clinical trials can also be used to determine the efficacy of a compound in humans.

The compositions may, if desired, be presented in a pack or dispenser device which may contain one or more unit dosage forms containing the active ingredient. The pack may for example comprise metal or plastic foil, such as a blister pack. The pack or dispenser device may be accompanied by instructions for administration. The pack or dispenser may also be accompanied with a notice associated with the container in form prescribed by a governmental agency regulating the manufacture, use, or sale of pharmaceuticals, which notice is reflective of approval by the agency of the form of the drug for human or veterinary administration. Such notice, for example, may be the labeling approved by the U.S. Food and Drug Administration for prescription drugs, or the approved product insert. Compositions comprising a compound of the subject technology Formulated in a compatible pharmaceutical carrier may also be prepared, placed in an appropriate container, and labeled for treatment of an indicated condition.

H. Control Release System

In another embodiment, the formulations may be a conventional controlled release system coated with or formulated to contain rate controlling materials. Such controlled release systems include, but are not limited to, gelatin capsules with enteric coatings, tablet formulations, and osmotic-pump-based delivery systems.

Positively charged biocompatible materials such as chitosan, poly(L-lysine), and poly(ethylene imines) are suitable for coating nanoparticles. Lectins may also be used to coat particles. Lectins may particularly target M cells in Peyer's patches in the intestine, enhancing the affinity of the particles for the intestinal wall. Lectins are produced by a wide variety of plants; one skilled in the art will recognize that not all lectins are appropriate for use in pharmaceutical compositions. A wide variety of lectins are available from Sigma-Aldrich, which also provides information on the toxicity and mutagenicity of commercially available lectins. One skilled in the art will recognize that lectins that are found in commonly eaten foods are more likely to be suitable for use with embodiments of the subject technology. Negatively charged materials may also be employed. Exemplary bioadhesive materials also include, without limitation, lecithin, polycarboxylic acids, poly(acrylic acids), polysaccharides, monosaccharides, oligosaccharides, oligopeptides, polypeptides, and co-polymers of two or more mucoadhesive materials. Alternatively or in addition, mucoadhesive or non-mucoadhesive polymers may be modified with mucoadhesive materials. For example, sugars may be covalently linked to polyacrylates. Additional bioadhesive molecules that may be used with the subject technology include but are not limited to hydrophilic and amphiphilic polymers, hydrogels, and the polymers disclosed in U.S. Pat. Nos. 6,217,908, 6,297,337; 6,514,535; and 6,284,235 the contents of which are incorporated herein by reference. Bioadhesive molecules may be PEGylated or otherwise modified as described above.

One skilled in the art will recognize that excessive cross-linking of the coating material may hinder release of the active agent from the core of the particle. The skilled artisan will also recognize that the effect of cross-linking may be easily tested by measuring the release of an active agent or a labeled analog from particles coated with materials having different degrees of cross-linking.

Examples of suitable coating materials include, but are not limited to, cellulose polymers such as cellulose acetate phthalate, hydroxypropyl cellulose, hydroxypropyl methylcellulose, hydroxypropyl methylcellulose phthalate and hydroxypropyl methylcellulose acetate succinate; polyvinyl acetate phthalate, acrylic acid polymers and copolymers, and methacrylic resins that are commercially available under the trade name EUDRAGIT® (Roehm Pharma, Darmstadt, Germany), zein, shellac, and polysaccharides. Additionally, the coating material may contain conventional carriers such as plasticizers, pigments, colorants, glidants, stabilization agents, pore formers and surfactants.

Examples of rate controlling polymers that may be used in the dosage form are hydroxypropylmethylcellulose (HPMC) with viscosities of either 5, 50, 100 or 4000 cps or blends of the different viscosities, ethylcellulose, methylmethacrylates, such as EUDRAGIT® RS100, EUDRAGIT® RL100, EUDRAGIT® NE 30D (supplied by Rohm America). Gastrosoluble polymers, such as EUDRAGIT® E100 or enteric polymers such as EUDRAGIT® L100-55D, L100 and 5100 may be blended with rate controlling polymers to achieve pH dependent release kinetics. Other hydrophilic polymers such as alginate, polyethylene oxide, carboxymethylcellulose, and hydroxyethylcellulose may be used as rate controlling polymers. Examples of suitable enteric coatings and the corresponding target region for release localized control are listed in Table 1.

TABLE 1
Examples of EUDRAGIT ® polymers for use as enteric polymers

| Enteric Coatings | Soluble pH | Target release region |
|---|---|---|
| EUDRAGIT L 100-55 | >5.5 | Duodenum |
| EUDRAGIT L 30 D-55 | >5.5 | Duodenum |
| EUDRAGIT L 100 | >6.0 | Jejunum-Ileum |
| EUDRAGIT L 100/S 100 | >6.5 | Ileum |
| EUDRAGIT S 100 | >7.0 | Colon |
| EUDRAGIT FS 30 D | >7.0 | Colon |
| EUDRAGIT L 12,5 | >6.0 | Jejunum |
| EUDRAGIT S 12,5 | >7.0 | Colon |
| EUDRAGIT NE 30 D | swellable | Duodenum-Jejunum |
| EUDRAGIT NE 40 D | swellable | Ileum-Colon |
| EUDRAGIT RL 30 D | swellable | Stomach |
| EUDRAGIT RL PO | swellable | Stomach |
| EUDRAGIT RL 100 | swellable | Ileum |
| EUDRAGIT RS 30 D | swellable | Duodenum-Colon |
| EUDRAGIT RS PO | swellable | Duodenum-Colon |
| EUDRAGIT RS 100 | swellable | Jejunum-Colon |
| EUDRAGIT E 100 | <5.0 | Stomach |
| EUDRAGIT E PO | <5.0 | Stomach |

Swellable EUDRAGIT ® is pH independent, time dependent.

I. Functionalized Polyoxalate Nanoparticles

In an embodiment, the polyoxalate nanoparticles of the subject technology include a functional moiety such as deoxyglucose (2-Deoxy-D-Glucose or 2-DG) which is a known tumor-targeting molecule. A functional moiety such as 2-DG can be directly or indirectly attached to the polyoxalate nanoparticles. A tumor-targeting molecule, for example, is any molecule that is more readily taken up by tumor cells than by non-tumor cells. A tumor-targeting molecule may be a chemical, a chemical group, a compound, an antibody, a nucleic acid, a peptide, a polypeptide, or the like. In some embodiments, the tumor-targeting molecule is a molecule, molecular group, or a compound. In certain aspects, the tumor-targeting compound is glucose or a glucose derivative. Glucose molecules useful in some embodiments include D-glucose and L-glucose. A glucose derivative refers to a molecule derived from glucose and includes any molecule derived from glucose, including any of the examples of glucose derivatives provided herein. Non-limiting examples of glucose derivatives include deoxyglucose, 2-Deoxy-D-Glucose, alpha-D-glucopyranose, beta-D-glucopyranose, 3-phospho-D-glycerate, alpha-D-glucose-1-phosphate, alpha-D-glucose-6-phosphate, beta-D-glucose-6-phosphate, beta-D-glucuronate, beta-D-glucosamine, beta-D-glucosamine-6-phosphate, D-glucosamine-6-phosphate, D-glucosamine, D-glucosaminide, D-glucosaminyl-D-glucosaminide, glucose-1,6-bisphosphate, glucose-1-phosphate, glucose-6-phosphate, and others known to those in the art. In certain aspects, the glucose derivative is deoxyglucose or 2-Deoxy-D-Glucose. In specific embodiments, a glucose derivative is attached directly to the nanoparticle. In other embodiments, a glucose derivative is attached indirectly to the nanoparticle, which means that another chemical moiety is attached directly to the nanoparticle and to the glucose derivative.

In some embodiments, subject 2DG-functionalized polyoxalate nanoparticles exhibit differential affinity for a particular mammalian tissue. In some embodiments, subject 2DG-functionalized polyoxalate nanoparticles exhibit differential affinity for a diseased mammalian tissue, e.g., subject 2DG-functionalized polyoxalate nanoparticles exhibit an affinity for a diseased tissue that is at least about 10%, at least about 25%, at least about 50%, at least about 100% (or 2-fold), at least about 2.5-fold, at least about 5-fold, at least about 10-fold, at least about 15-fold, at least about 20-fold, at least about 50-fold, or at least about 100-fold, or more, higher than the affinity of the 2DG-functionalized polyoxalate nanoparticles for a non-diseased tissue, e.g., for a non-diseased tissue of the same tissue type. For example, in some embodiments, subject 2DG-functionalized polyoxalate nanoparticles exhibit differential affinity for an epileptic brain tissue, e.g., subject 2DG-functionalized polyoxalate nanoparticles exhibit an affinity for an epileptic brain tissue that is at least about 10%, at least about 25%, at least about 50%, at least about 100% (or 2-fold), at least about 2.5-fold, at least about 5-fold, at least about 10-fold, at least about 15-fold, at least about 20-fold, at least about 50-fold, or at least about 100-fold, or more, higher than the affinity of the 2DG-functionalized polyoxalate nanoparticles for a non-epileptic brain tissue, e.g., for a non-epileptic brain tissue of the same brain region as the epileptic brain tissue.

In some embodiments, subject 2DG-functionalized polyoxalate nanoparticles exhibit differential metabolic uptake into a particular mammalian cell and/or tissue. In some embodiments, subject 2DG-functionalized polyoxalate nanoparticles exhibit differential metabolic uptake into a diseased mammalian fissile, e.g., subject 2DG-functionalized polyoxalate nanoparticles exhibit an at least about 1%, at least about 5%, at least about 10%, at least about 25%, at least about 50%, at least about 100% (or 2-fold), at least about 2.5-fold, at least about 5-fold, at least about 10-fold, at least about 15-fold, at least about 20-fold, at least about 50-fold, or at least about 100-fold, or more, greater metabolic uptake into the diseased tissue, compared to the metabolic uptake of the 2DG-functionalized polyoxalate nanoparticles into a non-diseased tissue, e.g., a non-diseased tissue of the same tissue type. For example, in some embodiments, subject 2DG-functionalized polyoxalate nanoparticles exhibit differential metabolic uptake into a cancerous mammalian tissue such as a tumor, e.g., subject 2DG-functionalized polyoxalate nanoparticles exhibit an at least about 1%, at least about 5%, at least about 10%, at least about 25%, at least about 50%, at least about 100% (or 2-fold), at least about 2.5-fold, at least about 5-fold, at least about 10-fold, at least about 15-fold, at least about 20-fold, at least about 50-fold, or at least about 100-fold, or more, greater metabolic uptake into the cancerous tissue (e.g., the tumor), compared to the metabolic uptake of the 2DG-functionalized, polyoxalate nanoparticles into a non-cancerous tissue, e.g., a non-cancerous tissue of the same tissue type, or compared to the metabolic uptake of the 2DG-functionalized polyoxalate nanoparticles into normal, non-cancerous tissue adjacent to or surrounding a tumor. Whether 2DG-functionalized polyoxalate nanoparticles exhibit differential metabolic uptake into a particular mammalian cell and/or tissue can be determined, e.g., using magnetic resonance imaging (MR1) or computed tomography (CT). A signal intensity change over time with repeated data acquisitions is observed when functionalized polyoxalate nanoparticles exhibit differential metabolic uptake into a cell and/or tissue.

### EXAMPLES

A better understanding of the subject technology may be obtained through the following examples which are set forth to illustrate, but are not to be construed as the limit of the subject technology.

### Example 1

### Polymerization of 1,4-Cyclohexanedimethanol and Oxalylchloride

To a solution of 1,4-cyclohexanedimethanol (CDE, 11.95 g) in methelene chloride (140 mL), pre-cooled oxalyl chloride (OC, 10.55 g) in methelene chloride (25 mL) was added drop wise while the CDE solution was set in an ice bath. After the OC was added into the CDE solution completely; a triethylamine (TEA, 30 mL) solution in methelene chloride (30 mL) was added drop wise. Then, the ice bath was removed and the mixture was stirred at room temperature overnight. Table 2 lists the various parameters used in this reaction.

TABLE 2
The parameters used in preparation of polyoxalate nanoparticles.

| No. | Polymer | DCM | PVA | Evaporation | Centrifuge |
|---|---|---|---|---|---|
| 1 | 0.5 g | 5 mL | 5 + 20 | S. at R.T | 11k/10 min. |
| 2 | 0.5 g | 10 mL | 5 + 20 | S. at R.T | 11k/10 min |
| 3 | 0.5 g | 0.5 mL | 5 + 20 | S. at R.T | 11k/10 min |
| 4 | 0.5 g | 5 mL | 2 + 20 | S. at R.T | 11k/10 min |
| 5 | 0.5 g | 10 mL | 2 + 20 | S. at R.T | 11k/10 min |
| 6 | 0.5 g | 0.5 mL | 10 + 20 | S. at R.T | 11k/10 min |
| 7 | 0.5 g | 5 mL | 10 + 20 | S. at R.T | 11k/10 min |
| 8 | 0.5 g | 10 mL | 10 + 20 | S. at R.T | 11k/10 min |
| 9 | 0.5 g | 10 mL | 0 + 20 | S. at R.T | 15k/30 min |

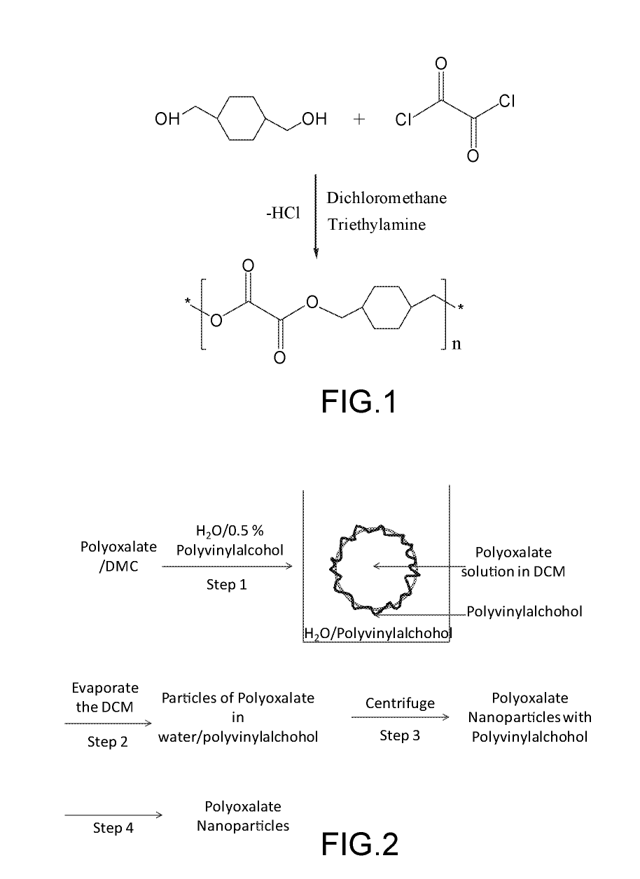

The mixture of reaction was extracted with methelene chloride (2×100 mL) after it was quenched with plenty of saturated brine. The organic extraction was washed with 0.1 N HCl (50 mL) to remove TEA. The organic phase was dried over MgSO₄ (20 g) overnight. After filtration and evaporation of the most methelene chloride, the residue was stirred with hexane at room temperature overnight. Then the solid was filtrated again and dried in vacuum oven. 15.2 g of the light yellow solid was obtained with about 92% yield. This high yield is stemmed to the step-by-step drop-wise addition of monomer oxalyl chloride and catalyst/neutralizer TEA. Following this procedure, the monomers added can react completely and hence result in a higher yield and higher polymerization degree (expected, polymerization degree not measured yet). The structure was confirmed by NMR. The chemical reaction is showed FIG. 1.

### Example 2

### Preparation of Polyoxalate Nanoparticles

To a solution of 0.5% polyvinylalcohol (PVA) solution (20 mL) in a beaker, a solution of 50 mg of polyoxalate polymer in 1 mL of methelene chloride was added drop wise with sonication and then for additional 2 minutes until a homogeneous emulsion formed. After the formation of homogenous emulsion, the organic solvent methelene chloride was evaporated by vigorous stirring in a hood. The resulting suspension was centrifuged at 18K rpm, 4° C. for 40 minutes. The clear solution was decanted and the residue was washed with water (3×5 mL). The solid was dried to get 20 mg of the white solid. The size of the nanoparticles prepared were about 108-140 nm in diameter measured by TEM. See also Table 3. The procedures are schematically shown in FIG. 2.

TABLE 3
Preparation of Polyoxalate nanoparticles with different sizes

| | | | | | Result |
|---|---|---|---|---|---|
| | | Mag. | | | (nm) (um/ |
| Sample | No | (K) | um/pix | pixel | pix × pix) |
| 1 | TS-03-10-14-01-16 | 6 | | | |
| | TS-03-10-14-05-59 | 10 | 0.004554 | 50 (L5) | ~230 |
| | TS-03-10-14-13-10 | 20 | 0.002432 | 51.42 (L8) | ~125 |
| | TS-03-10-14-24-33 | 20 | 0.002432 | 38 | 92.4 |
| | TS-03-10-14-25-56 | 20 | 0.002432 | | |
| 2 | TS-03-10-14-53-52 | 20 | 0.002432 | 30.27 (L3) | 73.6 |
| | TS-03-10-14-57-19 | 40 | 0.001176 | 74.32 (L2) | 87.4 |
| | TS-03-10-15-00-17 | 80 | 0.000662 | 208.47 | 138 |
| 3 | TS-03-10-15-16-24 | 20 | 0.002432 | 66.27 | 160 |

### Example 3

### Preparation of Polyoxalate Nanoparticles Loaded with Methazolamide

In this example, methazolamide-loaded nanoparticles were prepared.

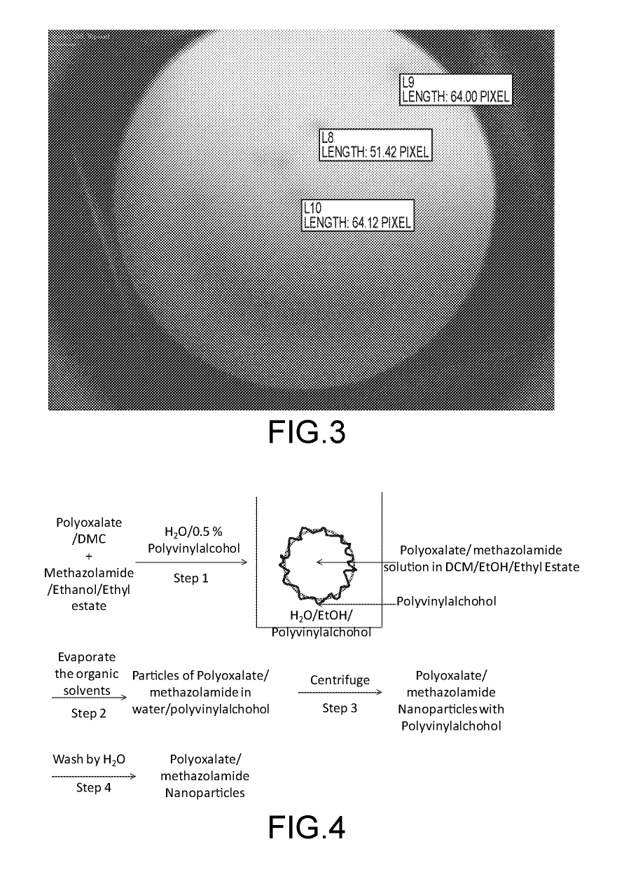

To a 0.5% PVA water solution (20 mL) in a beaker, a solution of 50 mg of polyoxalate polymer in 1 mL of methelene chloride and a solution of 5 mg of methazolamide in 6 mL of ethanol/ethyl acetate (1:5 in volume) were added under sonication for 5 minutes to form a homogeneous emulsion. Then, the organic solvents methelene chloride, ethyl acetate, and most ethanol were evaporated under vacuum. The resulting suspension was centrifuged at 18K rpm, 4° C. for 40 minutes. The clear solution was decanted and the residue was washed with water (3×5 mL). The solid was dried and about 20 mg of solid was obtained. The solid can be re-dispersed in water with gentle sonication. The size of the nanoparticles is about 263-274 nm in diameter as characterized by dynamic light scattering method. See FIG. 3. The procedure followed in this example is schematically shown in FIG. 4.

### Example 4

### Preparation of Polyoxalate Nanoparticles Loaded with CCK-8

This example discusses the preparation of CCK-8-loaded polyoxalate nanoparticles.

Briefly, 19.065 grams of cyclohexanediol was reacted with 13.6 grams of oxalyl chloride to produce 27.4 grams of the diacid. In a subsequent reaction, 8.1 grams of the diacid was reacted with 1 mL thionyl chloride followed by 0.098 grams of CCK-8 to furnish 7.4 grams of CCK-8-lodaded polyoxalate nanoparticles. The size of the CCK-8-loaded nanoparticles prepared were about 50 nm to about 300 nm in diameter (with a mean of about 100 nm to 200 nm) as characterized by dynamic light scattering method.

### Example 5

### Nanoparticles Uptake and Drug Release Studies in Mice

This example provides the results of an oral delivery of CCK-8-loaded nanoparticles of the subjection technology to mice. The profile of the CCK-8 release from these particles over time and the plasma CCK-8 concentrations after the oral delivery of the nanoparticles were also studied.

Materials and Methods: 4-6 (28-35 g) week old male ICR (CD-1®) Outbred Mice (Harlan Laboratories) were dosed by oral gavage, either CCK-8 loaded nanoparticles or empty nanoparticles and a timecourse of plasma CCK-8 concentration determined over 4 hrs. Mice were briefly anesthetized by exposure to 3% isoflurane in 100% oxygen for 1-2 minutes, and then maintained at 1.5% isoflurane in 100% oxygen (VetEquip IMPAC6 System). Mice were gavaged with 100 uL of nanoparticles, freshly suspended in PBS, pH 7.2 using a gently-curved silicon-tipped feeding needle (FST, Foster City, Calif.).

Mice were dosed in small batches to enable the suspended CCK-8-loaded nanoparticles to be freshly prepared and vortexed to a uniform suspension, immediately before use. In the experimental mouse set, the gavage volume of 100 μl, contained 66 μg of loaded nanoparticles with an equivalent cargo load of 15 μg CCK-8. In the unloaded nanoparticle group, mice were dosed with 63 μg of unloaded nanoparticles.

At selected timepoints (t=15, 30, 60, 120, 180 and 240 minutes), mice were euthanized by CO₂ inhalation, their abdominal and thorax cavities opened and 0.4-0.5 ml of blood was removed From their heart by cardiac puncture with a 2 ml syringe. The blood was transferred to a heparin/K2-EDTA microtainer tube (Becton-Dickinson) and immediately placed on ice. Blood samples were then spun down to separate the plasma for 10 min at 3,000 ref and the plasma removed and stored at −80° C. overnight.

Plasma CCK-8 concentrations were calculated using a mouse optimized CCK-8 ELISA assay (E91044Mu, USCN Life Sciences Inc. purchased from Antobodies Online.com, Atlanta, Ga.). The ELISA assay absorbance was measured at 450 nM in a 96 well plate Spectramax Plus (Molecular Devices, Sunnyvale, Calif.) spectrophotometer. Plasma CCK-8 concentrations were calculated against a standard curve, and data was plotted using Prizm 5 (Graphpad Software Inc., La Jolla, Calif.).

For CCK-8 loaded nanoparticles all data points were from 5 separate mice (total of 30 mice). For the empty nanoparticles, data points were from between 2 and 3 separate mice (total of 13 mice).

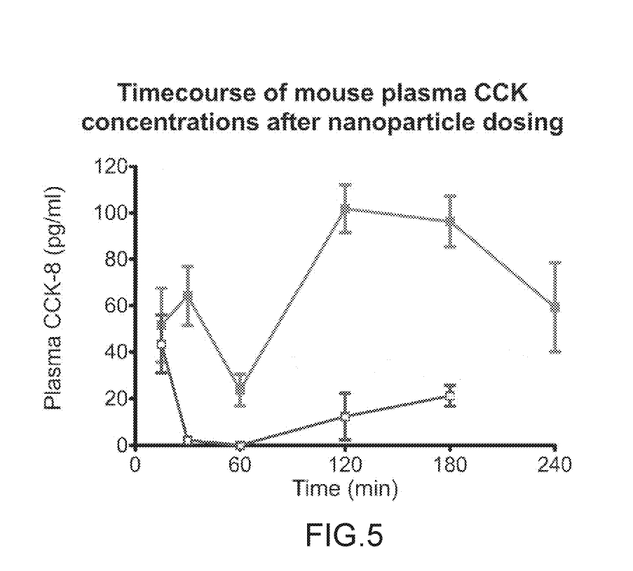

Results and Discussion: Initially (t=15 min) both experimental data sets show an elevated CCK-8 plasma concentration. See FIG. 5. Without being bound by any theories, it is hypothesized that initial elevation of the CCK-8 concentration was due to endogenous release from the gastric distention during gavage dosing. The plasma CCK-8 concentrations quickly decreased in the mice dosed with the empty nanoparticles, but was much slower to decrease in the CCK-8 loaded particle dosed mice. The plasma CCK-8 concentrations of the mice dosed with unloaded nanoparticles remains at baseline, less than 20 pg/ml from t=60 to 180 min. However, plasma concentration of CCK-8 continued to rise in the mice dosed orally with the loaded nanoparticles, which peaked at 120 min, as the particles were absorbed by the small intestine into the blood and CCK-8 release continued. The plasma CCK-8 levels in the mice released from the loaded nanoparticle group remained elevated when compared to control mice, even after 4 hrs.

These results show that, upon an oral delivery, the CCK-loaded polyoxalate nanoparticles of the subject technology can adhere to the intestinal lining and gradually pass through the lining into the circulatory system, where they can gradually release their payload of CCK at a rate determined by the decomposition rate of the core. These results also show that the CCK-loaded nanoparticles of the subject technology are capable of avoiding first pass hepatic metabolism (which occurs when drugs are removed from the bloodstream as they pass through the liver) and successfully reach the systemic circulation where they can release their payload.

### Example 6

### Functionalized Polyoxalate Nanoparticles

This example discusses the preparation of functionalized polyoxalate nanoparticles in which the functional moiety was 2-deoxyglucose (2-DG).

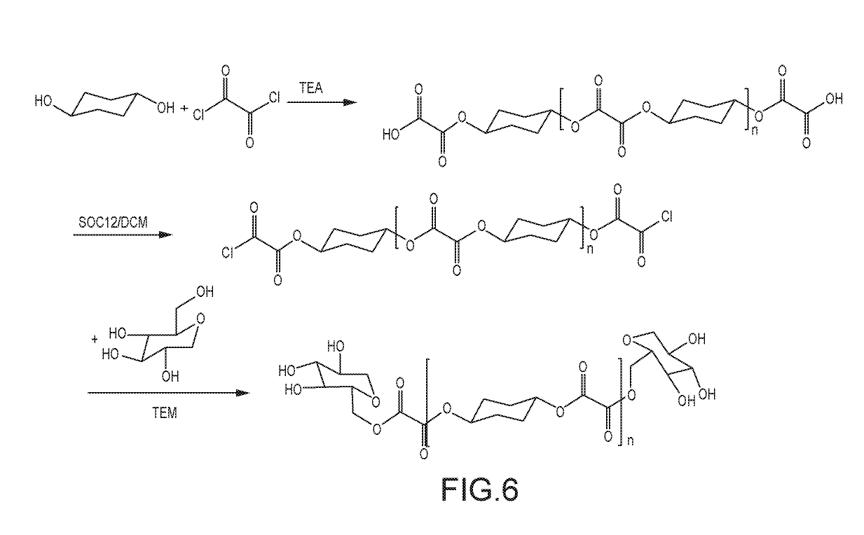

Briefly, 19.065 grams of cyclohexanediol was reacted with 13.6 grams of oxalyl chloride to produce 27.4 grams of the diacid. In a subsequent reaction, 8.1 grams of the diacid was reacted with 1 mL thionyl chloride followed by 0.098 grams of 2-deoxy-2-D-glucose to furnish 7.4 grams of deoxyglucose functionalized polymer. See FIG. 6.

### Example 7

### Functionalized Polyoxalate Nanoparticles Loaded with a Fluorescent Label

This example discusses preparation of 2-DG functionalized polyoxalate nanoparticles loaded with 4-dimethylamino-4′-nitrostilbene.

Briefly, 23 mg fluorescent label (stilbene) was dissolved into 2 mL dichloromethane to form a clear solution (1), while the corresponding deoxyglucose functionalized polymer (204 mg) was dissolved in a 10 mL of DCM to form a clear solution (11). The solution I and II were combined to form a solution III, which was added drop wise into. PVA 1 solution with sonification to form a homogeneous suspension. The suspension was added drop wise into PVA 2 solution. After complete homogenization, the suspension was stirred at room temperature to drive off DCM. Solid particles were obtained by centrifugation.

The foregoing description is provided to enable a person skilled in the art to practice the various configurations described herein. While the subject technology has been particularly described with reference to the various figures and configurations, it should be understood that these are for illustration purposes only and should not be taken as limiting the scope of the subject technology.

There may be many other ways to implement the subject technology. Various functions and elements described herein may be partitioned differently from those shown without departing from the scope of the subject technology. Various modifications to these configurations will be readily apparent to those skilled in the art, and generic principles defined herein may be applied to other configurations. Thus, many changes and modifications may be made to the subject technology, by one having ordinary skill in the art, without departing from the scope of the subject technology.

It is understood that the specific order or hierarchy of steps in the processes disclosed is an illustration of exemplary approaches. Based upon design preferences, it is understood that the specific order or hierarchy of steps in the processes may be rearranged. Some of the steps may be performed simultaneously. The accompanying method claims present elements of the various steps in a sample order, and are not meant to be limited to the specific order or hierarchy presented.

Although the subject technology has been described with reference to the examples provided above, it should be understood that various modifications can be made without departing from the spirit of the subject technology.

Throughout this application, underlined, italicized and/or boldface headings and subheadings are used for convenience only, do not limit the subject technology, and are not referred to in connection with the interpretation of the description of the subject technology. All structural and functional equivalents to the elements of the various configurations described throughout this disclosure that are known or later come to be known to those of ordinary skill in the art are expressly incorporated herein by reference and intended to be encompassed by the subject technology. Moreover, nothing disclosed herein is intended to be dedicated to the public regardless of whether such disclosure is explicitly recited in the above description.

What is claimed is:

1. Polyoxalate nanoparticles having diameters ranging from about 50 to about 300 nm.

2. A method for preparing biocompatible nanoparticles of polyoxalate polymers, comprising:
(a) mixing 1,4-cyclohexanedimethanol (CDE) with oxalyl chloride (OC) in the presence of dichloromethane (DCM), and triethylamine to produce polyoxalate polymer; and
(b) adding the polyoxalate polymer to a solution of polyvinylalcohol (PVA) in the presence of a suitable solvent to produce a substantially homogenous emulsion of polyoxalate nanoparticles.

3. The method of claim 2, wherein the produced polyoxalate nanoparticles have diameters ranging from about 50 to about 300 nm.

4. The method of claim 2, further comprising at least one of drying the nanoparticles or dispersing the nanoparticles in an aqueous solution.

5. The method of claim 2, wherein the suitable solvent comprises at least one of water, dichloromethane, acetonitrile, chloroform, dimethylformamide, tetrahydrofuran, ethanol, ethyl acetate, or acetone.

6. A method, for preparing polyoxalate nanoparticles for delivery of a drug, comprising:
(a) mixing 1,4-cyclohexanedimethanol (CDE) with oxalyl chloride (OC) in the presence of dichloromethane (DCM), and triethylamine to produce polyoxalate polymer;
(b) mixing the polyoxalate polymer with a drug in the presence of polyvinylalcohol and a suitable solvent to form a substantially homogeneous emulsion of polyoxalate nanoparticles containing the drug.

7. The method of claim 6, wherein the formed polyoxalate nanoparticles containing the drug have diameters ranging from about 50 to about 300 nm.

8. The method of claim 6, further comprising at least one of drying the nanoparticles containing the drug or dispersing the nanoparticles containing the drug in an aqueous solution.

9. The method of claim 6, further comprising solidifying the polyoxalate nanoparticles containing the drug at room temperature.

10. The method of claim 9, further comprising redispersing the solidified polymeric mixture in an aqueous solution.

11. The method of claim 6, wherein the suitable solvent comprises at least one of water, dichloromethane, acetonitrile, chloroform, dimethyl formamide, tetrahydrofuran, ethanol, ethyl acetate, or acetone.

12. Drug delivery nanoparticles comprising polyoxalate polymers, wherein the nanoparticles have diameters ranging from about 50 to about 300 nm.

13. The drug delivery nanoparticles of claim 12, wherein the nanoparticles comprise a payload.

14. The drug delivery nanoparticles of claim 13, wherein the payload comprises a therapeutic or diagnostic agent.

15. The drug delivery nanoparticles of claim 14, wherein the therapeutic agent is selected from the group consisting of methazolamide, insulin and cholecystokinin (CCK).

16. The drug delivery nanoparticles of claim 15, wherein the cholecystokinin is any one of cholecystokinin-8 (CCK-8), N-sarkosyl-CCK-8, N-taurine-CCK-8, N-pyroglutamic-CCK-8, C-terminal heptapeptide of CCK (CCK-7), N-sarkosyl-CCK-7, N-taurine-CCK-7, N-pyroglutamic-CCK-7, t-BOCK-CCK-7 or cholecystokinin-4 (CCK-4).

17. A composition comprising drug delivery nanoparticles comprising polyoxalate polymers, wherein the nanoparticles have diameters ranging from about 50 to about 300 nm.

18. The composition of claim 17, wherein the nanoparticles further comprise a payload.

19. The composition of claim 18, wherein the payload comprises a therapeutic or diagnostic agent.

20. The composition of claim 19, wherein the therapeutic agent is selected from the group consisting of methazolamide, insulin and cholecystokinin (CCK).

21. The composition of claim 20, wherein the cholecystokinin (CCK) is any one of cholecystokinin-8 (CCK-8), N-sarkosyl-CCK-8, N-taurine-CCK-8, N-pyroglutamic-CCK-8, C-terminal heptapeptide of CCK (CCK-7), N-sarkosyl-CCK-7, N-taurine-CCK-7, N-pyroglutamic-CCK-7, t-BOCK-CCK-7 or cholecystokinin-4 (CCK-4).

22. Drug delivery nanoparticles comprising polyoxalate polymers, wherein the nanoparticles have diameters ranging from about 50 to about 300 nm, and wherein the nanoparticles further comprise a functional moiety.

23. The drug delivery nanoparticles of claim of claim 22, wherein the functional moiety comprises 2-deoxyglucose.


SEQUENCE LISTING (2 sequences)

SEQ ID NO: 1 - protein, 4 aa, Artificial Sequence
  Description of Artificial Sequence Synthetic peptide
WMDF

SEQ ID NO: 2 - protein, 8 aa, Artificial Sequence [sequence omitted]